SEQUENCE LISTING (37 sequences)

SEQ ID NO: 1 - protein, 5 aa, Mus musculus
  MISC_FEATURE (1)..(1): X is Absent or Thr
XNNLX

SEQ ID NO: 2 - protein, 8 aa, Mus musculus
INPGSGDT

SEQ ID NO: 3 - protein, 11 aa, Mus musculus
GDYYGGFAVDY

SEQ ID NO: 4 - protein, 6 aa, Mus musculus
QDIGNY

SEQ ID NO: 5 - protein, 4 aa, Mus musculus
YTSR

SEQ ID NO: 6 - protein, 7 aa, Mus musculus
QQGKSLP

SEQ ID NO: 7 - protein, 5 aa, Mus musculus
NNLIE

SEQ ID NO: 8 - protein, 17 aa, Mus musculus
VINPGSGDTNYNEKFKG

SEQ ID NO: 9 - protein, 11 aa, Mus musculus
GDYYGGFAVDY

SEQ ID NO: 10 - protein, 11 aa, Mus musculus
RTSQDIGNYLN

SEQ ID NO: 11 - protein, 7 aa, Mus musculus
YTSRLHS

SEQ ID NO: 12 - protein, 7 aa, Mus musculus
QQGKSLP

SEQ ID NO: 13 - protein, 8 aa, Mus musculus
GYAFTNNL

SEQ ID NO: 14 - protein, 8 aa, Mus musculus
INPGSGDT

SEQ ID NO: 15 - protein, 13 aa, Mus musculus
ARGDYYGGFAVDY

SEQ ID NO: 16 - protein, 6 aa, Mus musculus
QDIGNY

SEQ ID NO: 17 - protein, 4 aa, Mus musculus
YTSR

SEQ ID NO: 18 - protein, 9 aa, Mus musculus
QQGKSLPRT

SEQ ID NO: 19 - protein, 10 aa, Mus musculus
GYAFTNNLIE

SEQ ID NO: 20 - protein, 17 aa, Mus musculus
VINPGSGDTNYNEKFKG

SEQ ID NO: 21 - protein, 13 aa, Mus musculus
ARGDYYGGFAVDY

SEQ ID NO: 22 - protein, 11 aa, Mus musculus
RTSQDIGNYLN

SEQ ID NO: 23 - protein, 7 aa, Mus musculus
YTSRLHS

SEQ ID NO: 24 - protein, 9 aa, Mus musculus
QQGKSLPRT

SEQ ID NO: 25 - DNA, 467 bp, artificial sequence
  Recombinant DNA
atgggatggaccttggtctttctctttctcctgtcagtaactgcaggtgttcactcccaggtccagctgcagcagtctggagctgagctggtaaggcctgggacttcagtgaaggtgtcctgcaaggcttctggatacgccttcactaataacttgatagagtgggtaaaacagaggcctggacagggccttgagtggattggagtgattaatcctggaagtggtgatactaactacaatgagaagttcaagggcaaggcaacactgactgcagacaaatcctccaacactgcctacatgcagctcagcagcctgacatctgatgactctgcggtctatttctgtgcaagaggggattactacggtggctttgctgtggactactggggtcaaggaacctcagtcaccgtctcctcagccaaaacgacacccccatccgtttatcccttggcccctggaagcttggg

SEQ ID NO: 26 - protein, 120 aa, artificial sequence
  Recombinant polypeptide
QVQLQQSGAELVRPGTSVKVSCKASGYAFTNNLIEWVKQRPGQGLEWIGVINPGSGDTNYNEKFKGKATLTADKSSNTAYMQLSSLTSDDSAVYFCARGDYYGGFAVDYWGQGTSVTVSS

SEQ ID NO: 27 - DNA, 488 bp, artificial sequence
  Recombinant DNA
atggtgtcctcagctcagttccttggtctcctgttgctctgttttcaaggaaccagatgtgatatccagatgacacagactacatcctccctgtctgcctctctgggagacagagtcaccatcagttgcaggacaagtcaggacattggcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaaaagccttcctcggacgttcggtggaggcaccaagttggaaatcaaacgggctgatgctgcaccaactgtatccatcttcccaccatccagtgagcagttaacatctggaggtgcctcagtcgtgtgcttcttgaacaacttctaccccagaga

SEQ ID NO: 28 - protein, 107 aa, artificial sequence
  Recombinant polypeptide
DIQMTQTTSSLSASLGDRVTISCRTSQDIGNYLNWYQQKPDGTVKLLIYYTSRLHSGVPSRFSGSGSGTDYSLTISNLEQEDIATYFCQQGKSLPRTFGGGTKLEIK

SEQ ID NO: 29 - protein, 10 aa, artificial sequence
  Recombinant peptide
DIQMTQTTSS

SEQ ID NO: 30 - protein, 450 aa, artificial sequence
  Recombinant polypeptide
QVQLQQSGAELVRPGTSVKVSCKASGYAFTNNLIEWVKQRPGQGLEWIGVINPGSGDTNYNEKFKGKATLTADKSSNTAYMQLSSLTSDDSAVYFCARGDYYGGFAVDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 214 aa, artificial sequence
  Recombinant polypeptide
DIQMTQTTSSLSASLGDRVTISCRTSQDIGNYLNWYQQKPDGTVKLLIYYTSRLHSGVPSRFSGSGSGTDYSLTISNLEQEDIATYFCQQGKSLPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 13 aa, Homo sapiens
VLLLVHNLPQQLF

SEQ ID NO: 33 - protein, 12 aa, Homo sapiens
YPNASLLIQNVT

SEQ ID NO: 34 - protein, 7 aa, Homo sapiens
PNASLLI

SEQ ID NO: 35 - protein, 766 aa, Homo sapiens [sequence omitted]

SEQ ID NO: 36 - protein, 393 aa, Homo sapiens [sequence omitted]

SEQ ID NO: 37 - protein, 400 aa, Homo sapiens [sequence omitted]

(19) United States
(12) Patent Application Publication
(10) Pub. No.: US 2016/0376374 A1
(43) Pub. Date: Dec. 29, 2016

(54) ANTIBODIES TO CEACAM1 AND KINASE INHIBITORS FOR TREATING BRAF-MUTATED CELLS

(71) Applicant: TEL HASHOMER MEDICAL RESEARCH INFRASTRUCTURE AND SERVICES LTD., Ramat Gan (IL)

(72) Inventors: Gal Markel, Ramat Gan (IL); Jacob Schachter, Givataim (IL)

(73) Assignee: TEL HASHOMER MEDICAL RESEARCH INFRASTRUCTURE AND SERVICES LTD., Ramat Gan (IL)

(21) Appl. No.: 15/109,182

(22) PCT Filed: Jan. 1, 2015
(86) PCT No.: PCT/IL2015/050007

§ 371 Date: Jun. 30, 2016

Related U.S. Application Data
(60) Provisional application No. 61/922,879, filed on Jan. 2, 2014.

(51) Int. Cl. C07K 16/30; A61K 39/395; C12Q 1/68; A61K 31/437; A61K 31/4184; C07K 16/28; A61K 45/06

(52) U.S. Cl. CPC C07K 16/3007; C07K 16/2803; A61K 39/39558; A61K 45/06; A61K 31/437; A61K 31/4184; C12Q 1/6886; C07K 2317/92; C07K 2317/565; C12Q 2600/156; C12Q 2600/106; C12Q 2600/158

(57) ABSTRACT

Pharmaceutical compositions comprising anti-CEACAM1 antibodies and kinase inhibitors are provided as well as methods for their use in treating cancer.

### FIELD OF THE INVENTION

The present invention relates to combinations of kinase inhibitors and antibodies to CEACAM1, and to their use in treating cancer.

### BACKGROUND OF THE INVENTION

The transmembrane protein carcinoembryonic antigen-related cell adhesion molecule 1 (CEACAM1, also known as biliary glycoprotein (BGP), CD66a and C-CAM1), is a member of the carcinoembryonic antigen family (CEA) that also belongs to the immunoglobulin superfamily. Human CEACAM1 has been assigned the SwissProt accession number P13688. CEACAM1 interacts with itself and with other known CEACAM proteins, including CD66c (CEACAM6) and CD66e (CEACAM5, CEA) proteins. It is expressed on a wide spectrum of cells, ranging from epithelial cells to those of hemopoietic origin (e.g. immune cells).

Many different functions have been attributed to the CEACAM1 protein. It was shown that the CEACAM1 protein is over expressed in some carcinomas of colon, prostate, as well as other types of cancer, such as melanoma. Additional data support the central involvement of CEACAM1 in angiogenesis and metastasis. CEACAM1 also plays a role in the modulation of innate and adaptive immune responses. For example, CEACAM1 was shown to be an inhibitory receptor for activated T cells contained within the human intestinal epithelium (WO 99/52552 and Morales et al. J. Immunol. 1999, 163, 1363-1370). Additional reports have indicated that CEACAM1 engagement either by T cell receptor cross-linking with monoclonal antibodies (mAbs) inhibits T cell activation and proliferation. Several monoclonal antibodies against the CEACAM1 protein are already known, such as 26H7, 5F4, TEC-11, 12-140-4, 4/3/17, COL-4, F36-54, 34B1, YG-C28F2, D14HD11, b18.7.7, D11-AD11, HEA81, B1.1, CLB-gran-10, F34-187, T84.1, B6.2, B1.13, YG-C94G7, 12-140-5, TET-2 and scFv-DIATHIS1 (Watt et al., Blood, 2001, Vol. 98, pages 1469-1479). WO 2010/12557 describes a murine monoclonal antibody to human CEACAM1. WO 2013/054331 describes a chimeric monoclonal antibody to human CEACAM1.

Mitogen-activated protein kinase (MAPK) is an important signaling pathway in humans. When stimulated, factors of upstream or downstream change, and by interacting with each other, these factors have long been recognized to be related to multiple biologic processes such as cell proliferation, differentiation, death, migration, invasion and inflammation. However, once abnormally activated, cancer may occur. Several components, such as RAF and MEK, of the MAPK network have already been proposed as targets in cancer therapy.

There are three RAF paralogs in humans: A-Raf, B-Raf, and C-Raf. These serine/threonine protein kinases are components of a conserved signaling pathway downstream of the membrane-bound small G protein RAS, which is activated by growth factors, hormones and cytokines. RAS stimulates RAF activation, which then activates a second protein kinase called MEK, which in turn activates a third protein kinase called ERK. ERK regulates gene expression, cytoskeletal rearrangements, and metabolism to coordinate responses to extracellular signals and regulate proliferation, differentiation, senescence, and apoptosis. This pathway is hyper-activated in about 30% of cancers with activating mutations in RAS occurring in approximately 15%-30% of cancers. Data has shown that B-Raf is mutated in about 7% of all cancers, identifying it as another important oncogene on this pathway (Garnett and Marais, Cancer Cell, 2004, Vol. 6, pages 313-319). The highest incidence of B-Rad mutation is in malignant melanoma (27-70%).

The gene BRAF encodes the serine/threonine-protein kinase B-Raf (UniProt P15056), which is involved in sending signals inside cells, which are involved in directing cell growth. Drugs that treat cancers driven by BRAF mutations have been developed. Two of these drugs, vemurafenib (N-(3-{[5-(4-chlorophenyl)-1H-pyrrolo[2,3-b]pyridin-3-yl]carbonyl}-2,4-difluorophenyl)propane-1-sulfonamide) and dabrafenib (N-{3-[5-(2-aminopyrimidin-4-yl)-2-tert-butyl-1,3-thiazol-4-yl]-2-fluorophenyl}-2,6-difluoro-benzenesulfonamide) are approved by FDA for treatment of late-stage melanoma.

Mitogen-activated protein kinase, or Mitogen/Extracellular signal-regulated kinase (also known as MAP2K, MEK, MAPKK) is a kinase enzyme which phosphorylates mitogen-activated protein kinase (MAPK). There are seven genes in this family: MAP2K1 (also known as MEK1), MAP2K2 (MEK2), MAP2K3 (MKK3), MAP2K4 (MKK4), MAP2K5 (MKK5), MAP2K6 (MKK6) and MAP2K7 (MKK7). The activators of p38 (MKK3 and MKK6), JNK (MKK4 and MKK7), and ERK (MEK1 and MEK2), that define independent MAP kinase signal transduction pathways.

MEK inhibitors inhibit the mitogen-activated protein kinase kinase enzymes MEK1 and/or MEK2. They can be used to affect the MAPK/ERK pathway which is often overactive in some cancers. Some MEK inhibitors are trametinib (N-(3-{3-Cyclopropyl-5-[(2-fluoro-4-iodophenyl)amino]-6,8-dimethyl-2,4,7-trioxo-3,4,6,7-tetra-hydropyrido[4,3-d]pyrimidin-1-(2H)-yl}phenyl)acetamide), FDA-approved to treat B-Raf-mutated melanoma, and selumetinib (6-(4-bromo-2-chloroanilino)-7-fluoro-N-(2-hydroxyethoxy)-3-methylbenzimid-azole-5-carboxamide), an investigational drug.

Targeted therapy with B-Raf inhibitors was shown to be very effective, with high response rates and objective regression even in patients with very high tumor burdens. However, the main disadvantage of this therapy is recurrence of disease, typically occurring around 7-10 months post initiation. Recurrence occurs in almost all patients due to the development of resistance and reactivation of the MAPK pathway (Villanueva et al., Cancer Res., 2011, Vol. 71(23), pages 7137-7140).

There is an unmet need for providing additional and improved cancer therapies, including combination therapies.

### SUMMARY OF THE INVENTION

The present invention provides compositions and methods for treating cancer types associated with BRAF mutations. More specifically, the present invention provides compositions comprising antibodies to CEACAM1 in combination with inhibitors of the RAF/MEK pathway, and their use in treating cancers associated with B-Raf mutants having aberrant constitutive activity.

The present invention is based in part on the surprising findings that melanoma cells, while being treated with inhibitors of the RAF/MEK pathway, express low levels of CEACAM1, and that these cells, upon becoming resistant to the RAF/MEK inhibitors, express high levels of CEACAM1, higher than expressed by RAF/MEK inhibitor-naïve cancer cells. To avoid or minimize the emergence of such “escape mutants”, compositions and methods for combined anti-cancer therapy are provided.

The present invention thus provides, in one aspect, a pharmaceutical composition comprising an inhibitor of a kinase selected from the group consisting of a B-Raf kinase mutant, a MEK1 kinase and a MEK2 kinase, and a monoclonal antibody to human CEACAM1 or an antigen-binding fragment thereof.

In certain embodiments, the B-Raf kinase mutant is constitutively active. In certain embodiments, the B-Raf kinase inhibitor attenuates or prevents the phosphorylation of MEK1 or MEK2 by the B-Raf kinase mutant. In certain embodiments, the B-Raf kinase inhibitor attenuates or prevents the dimerization of the B-Raf kinase mutant. In certain embodiments, the MEK1 kinase inhibitor attenuates or prevents the phosphorylation of MAPK by the MEK1 kinase. In certain embodiments, the MEK2 kinase inhibitor attenuates or prevents the phosphorylation of MAPK by the MEK2 kinase.

The present invention further provides, in an aspect, an inhibitor of a kinase selected from the group consisting of a B-Raf kinase mutant, a MEK1 kinase and a MEK2 kinase, and a monoclonal antibody to human CEACAM1 or an antigen-binding fragment thereof, for use in treating cancer by separate or simultaneous administration.

The present invention further provides, in another aspect, the use of an inhibitor of a kinase selected from the group consisting of a B-Raf kinase mutant, a MEK1 kinase and a MEK2 kinase, and a monoclonal antibody to human CEACAM1 or an antigen-binding fragment thereof, in preparing a medicament for treating cancer.

The present invention thus provides, in yet another aspect, a pharmaceutical composition comprising a monoclonal antibody to human carcinoembryonic antigen-related cell adhesion molecule 1 (CEACAM1) or an antigen-binding fragment thereof, and a pharmaceutical composition comprising an inhibitor of a kinase selected from the group consisting of a B-Raf kinase mutant, a MEK1 kinase and a MEK2 kinase, for use in treatment of cancer by separate administration. The present invention also provides, in another aspect, a method of treating a patient having cancer, comprising the step of administering to the patient a pharmaceutical composition comprising an inhibitor of a kinase selected from the group consisting of B-Raf kinase mutant, a MEK1 kinase and a MEK2 kinase, and a pharmaceutical composition comprising a monoclonal antibody to human CEACAM1 or an antigen-binding fragment thereof, wherein the cancer cells express a B-Raf kinase mutant, thereby treating the cancer.

In certain embodiments, the monoclonal antibody to human CEACAM1 is capable of preventing, interfering or dissociating an interaction between CEACAM1 presented by lymphocytes and CEACAM1 presented by cancer cells. In certain embodiments, treating cancer means attenuating tumor progression, inhibiting the spread of the malignant cells in the patient, causing the death of malignant cells in the patient and any combination thereof. In certain embodiments, treating cancer means attenuating tumor progression, causing the death of malignant cells in the patient, or both. In some embodiments, treatment comprises reducing the size, maintaining the size or slowing the growth of a primary or metastatic tumor. In some embodiments, prevention of cancer comprises avoiding, postponing, or minimizing the appearance of a primary or a metastatic tumor. Each possibility represents a separate embodiment of the present invention.

The present invention further provides, in an aspect, a kit comprising an inhibitor of a kinase selected from the group consisting of a B-Raf kinase mutant, a MEK1 kinase and a MEK2 kinase, and a monoclonal antibody to human CEACAM1 or an antigen-binding fragment thereof. The present invention further provides, in an aspect, the kit described above, for use in treating cancer. The present invention further provides, in an aspect, the kit described above, for use in preparing a medicament for treating cancer.

Further, the present invention provides, in an aspect, a method of diagnosing resistance to an inhibitor of a kinase selected from the group consisting of a B-Raf kinase mutant, a MEK1 kinase and a MEK2 kinase in a cancer patient treated by the kinase inhibitor, comprising the steps of obtaining a biopsy sample from the patient, determining the level of CEACAM1 in cancer cells of the biopsy sample, and comparing the level of CEACAM1 in cancer cells of the biopsy sample to a predetermined threshold, wherein the cancer cells express a B-Raf kinase mutant, and wherein a level of CEACAM1 in cancer cells of the biopsy sample higher than the predetermined threshold is indicative of resistance to the kinase inhibitor in the patient.

The present invention further provides, in a related aspect, a method of diagnosing resistance to an inhibitor of a kinase selected from the group consisting of a B-Raf kinase mutant, a MEK1 kinase and a MEK2 kinase in a cancer patient treated by the kinase inhibitor, comprising the steps of determining the level of CEACAM1 in cancer cells of a biopsy sample obtained or derived from the patient, and comparing the level of CEACAM1 in cancer cells of the biopsy sample to a predetermined threshold, wherein the cancer cells express a B-Raf kinase mutant, and wherein a level of CEACAM1 in cancer cells of the biopsy sample higher than the predetermined threshold is indicative of resistance to the kinase inhibitor in the patient.

In certain embodiment, the predetermined threshold is the level of CEACAM1 in cells of a corresponding biopsy sample obtained or derived from a corresponding tissue of a healthy control subject. In certain embodiment, the predetermined threshold is the level of CEACAM1 in cells of a prior biopsy sample obtained or derived from the same tissue of the patient before the patient was treated with the kinase inhibitor. In some embodiments, the biopsy sample is obtained or derived from a nevus of the patient. In some embodiments, the biopsy sample is obtained or derived from a primary tumor of the patient. In some embodiments, the biopsy sample is obtained or derived from a metastatic tumor of the patient.

In certain embodiments, the pharmaceutical compositions described above are for use in treating a cancer selected from the group consisting of melanoma, thyroid, colon, ovarian, liver, sarcoma, stomach, glioma, carcinoma, breast, ependymoma and lung cancer. Each possibility represents a separate embodiment of the invention. In certain embodiments, the pharmaceutical compositions described above are for use in treating melanoma. In certain embodiments, the pharmaceutical compositions described above are for use in treating stage III cancer. In certain embodiments, the pharmaceutical compositions described above are for use in treating stage IV cancer.

In certain embodiments, the kinase inhibitor and the monoclonal antibody to human CEACAM1 or the antigen-binding fragment thereof are administered simultaneously. In certain embodiments, the kinase inhibitor and the monoclonal antibody to human CEACAM1 or the antigen-binding fragment thereof are comprised in the same pharmaceutical composition. In certain embodiments, the kinase inhibitor and the monoclonal antibody to human CEACAM1 or the antigen-binding fragment thereof are comprised in at least two separate pharmaceutical compositions.

In certain embodiments, the kinase inhibitor and the monoclonal antibody to human CEACAM1 or the antigen-binding fragment thereof are administered separately. In certain embodiments, the kinase inhibitor is administered prior to the administration of the monoclonal antibody to human CEACAM1 or the antigen-binding fragment thereof. In certain embodiments, the monoclonal antibody to human CEACAM1 or the antigen-binding fragment thereof is administered prior to the administration of the kinase inhibitor.

In certain embodiment, the B-Raf kinase inhibitor attenuates or prevents the phosphorylation of MEK1 or MEK2 by the B-Raf kinase in the cancer cells of the cancer patient. In certain embodiments, the B-Raf kinase inhibitor attenuates or prevents the dimerization of the B-Raf kinase mutant in the cancer cells of the cancer patient.

In certain embodiment, the methods described above further comprise the step of a surgical resection of the primary tumor. In certain embodiment, the methods described above further comprise the step of administering a chemotherapy agent to the patient. In certain embodiment, the method described above further comprises the step of exposing the patient to irradiation.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 presents the correlation between CEACAM1 expression and the presence of B-Raf V600E mutation in cancer cells.

FIG. 2 is an immunoblotting assay measuring MAPK activity by detection of phosphorylated ERK in B-Raf V600E cancer cells and B-Raf W.T. cancer cells after exposure to vemurafenib.

FIG. 3A-C is a flow cytometry assay demonstrating the effect of vemurafen-Raf W.T. cancer cells.

FIG. 4A-B presents the correlation between CEACAM1 expression and resistance to inhibitors of B-Raf mutants in cancer cells.

FIG. 5 is a flow cytometry assay demonstrating that vemurafenib-resistant B-Raf V600E cancer cells upregulate CEACAM1 expression.

FIG. 6A-B presents the correlation between expression of CEACAM1 types and resistance to inhibitors of B-Raf mutants in cancer cells.

FIG. 7A-B presents the effect of B-Raf inhibitors on the cytotoxicity of T cells towards B-Raf-mutated cancer cells.

### DETAILED DESCRIPTION OF THE INVENTION

The present invention relates to therapeutic combinations and treatment regimens of inhibitors of B-Raf, MEK1, or MEK2, and antibodies to human CEACAM1 molecules. The present invention further relates to methods for treating cancer employing such combinations.

The present invention stems from the surprising finding that there is a significant correlation between the appearance of a mutation in the human BRAF gene, the expression of a mutated B-Raf kinase and the expression level of CEACAM1 in cancer cells. The present invention further stems from the surprising finding that there is a significant correlation between the appearance of resistance of cancer cells to inhibitors of the RAF-MEK pathway and the expression level of CEACAM1 in these cancer cells.

More specifically, it has been surprisingly found that cancer cells expressing constitutively active forms of the B-Raf kinase co-express elevated level of CEACAM1 compared to normal, healthy cells. It has been further surprisingly found that these cancer cells, while being treated with inhibitors of the B-Raf/MEK1/MEK2 pathway, expressed reduced levels of CEACAM1 and that these cancer cells, upon acquiring resistance to these inhibitors of the B-Raf/MEK1/MEK2 pathway, once again expressed elevated levels of CEACAM1, higher than those expressed before being treated with these inhibitors. Taken together, this thus-far undescribed and surprising correlation between cancer cells' CEACAM1 levels and sensitivity to inhibitors of the B-Raf/MEK1/MEK2 pathway, forms the basis for the present invention, which provides compositions and methods for treating cancers carrying BRAF mutations and co-expressing B-Raf mutants and elevated levels of CEACAM1, and for diagnosing resistance to of B-Raf/MEK1/MEK2 inhibitors in these cells.

The present invention thus provides, in an aspect, a pharmaceutical composition comprising an inhibitor of a kinase selected from the group consisting of a B-Raf kinase mutant, a MEK1 kinase and a MEK2 kinase, and a monoclonal antibody to human CEACAM1 or an antigen-binding fragment thereof.

The present invention further provides, in an aspect, an inhibitor of a kinase selected from the group consisting of a B-Raf kinase mutant, a MEK1 kinase and a MEK2 kinase, and a monoclonal antibody to human CEACAM1 or an antigen-binding fragment thereof, for use in treating cancer.

The present invention further provides, in another aspect, the use of an inhibitor of a kinase selected from the group consisting of a B-Raf kinase mutant, a MEK1 kinase and a MEK2 kinase, and a monoclonal antibody to human CEACAM1 or an antigen-binding fragment thereof, in preparing a medicament for treating cancer.

The term “pharmaceutical composition” as used herein refers to a composition comprising at least one biologically active ingredient. Antibodies, antigen-binding fragments thereof, B-Raf kinase inhibitors, MEK1 kinase inhibitors and MEK2 kinase inhibitors are non-limiting examples of biologically active ingredients.

The term “B-Raf kinase mutant” or “mutant B-Raf kinase” refers to any B-Raf kinase protein carrying at least one amino-acid mutation compared to the human wild-type (W.T.) 766 amino-acid B-Raf kinase protein (UniProt P15056, serine/threonine-protein kinase B-raf), the amino-acid sequence thereof is set forth in SEQ ID NO: 35. In certain embodiments, the mutant B-Raf kinase comprises 1-20 mutations. In certain embodiments, the mutant B-Raf kinase comprises 1-10 mutations. In certain embodiments, the mutant B-Raf kinase comprises 1-5 mutations. In certain embodiments, the mutant B-Raf kinase comprises 1 mutation. Each possibility represents a separate embodiment of the present invention.

The term “inhibitor of a B-Raf kinase mutant” or “mutant B-Raf kinase inhibitor” or “B-Raf kinase mutant inhibitor” refers to any compound capable of interacting with a B-Raf kinase protein carrying at least one mutation compared to the human wild-type (W.T.) 766 amino-acid B-Raf kinase protein (UniProt P15056), the amino-acid sequence thereof is set forth in SEQ ID NO: 35, and decreasing or eliminating its kinase activity.

The term “inhibitor of a MEK1 kinase” or “MEK1 kinase inhibitor” refers to any compound capable of interacting with the human wild-type (W.T.) 393 amino-acid MEK1 kinase protein (UniProt Q02750, dual specificity mitogen-activated protein kinase kinase 1), the amino-acid sequence thereof is set forth in SEQ ID NO: 36, and decrease or eliminate its kinase activity.

The term “inhibitor of a MEK2 kinase” or “MEK2 kinase inhibitor” refers to any compound capable of interacting with the human wild-type (W.T.) 400 amino-acid MEK2 kinase protein (UniProt P36507, dual specificity mitogen-activated protein kinase kinase 2), the amino-acid sequence thereof is set forth in SEQ ID NO: 37, and decrease or eliminate its kinase activity.

The term “antibody” is used in the broadest sense and includes monoclonal antibodies (including full length or intact monoclonal antibodies), polyclonal antibodies, multivalent antibodies, multispecific antibodies (e.g., bispecific antibodies), immuno-modulatory agents, and antibody fragments of sufficient size to retain and exhibit the full antibody's desired biological activity.

The term “immuno-modulatory agent” or “immuno-modulatory protein” or “antibody fragment” as used interchangeably includes synthetic or genetically engineered proteins that act like an antibody by binding to a specific antigen to form a complex. For example, antibody fragments include isolated fragments consisting of the variable regions, such as the “Fv” fragments consisting of the variable regions of the heavy and light chains and recombinant single chain polypeptide molecules in which light and heavy variable regions are connected by a peptide linker (“scFv proteins”). The fragments may be constructed in different ways to yield multivalent and/or multi-specific binding forms. Antibody or antibodies according to the invention include intact antibodies, such as polyclonal antibodies or monoclonal antibodies (mAbs), as well as proteolytic fragments thereof such as the Fc, Fab or F(ab′)₂ fragments. Further included within the scope of the invention are chimeric antibodies; human and humanized antibodies; recombinant and engineered antibodies, and fragments thereof.

An “a monoclonal antibody to human CEACAM1”, “an antibody which recognizes CEACAM1”, “an antibody against CEACAM1”, or “an antibody to CEACAM1” is an antibody that binds to the CEACAM1 protein with sufficient affinity and specificity. Typically, a monoclonal antibody to human CEACAM1 is capable of binding CEACAM1 with a minimal affinity of about 10⁻⁸ or 10⁻⁹ M. Some of the monoclonal anti-CEACAM1 antibodies are capable of binding CEACAM3, 5 and/or 8 with a minimal affinity of about 5×10⁻⁷ M. In certain embodiments, the monoclonal antibody to human CEACAM1 is capable of preventing, interfering or dissociating an interaction between CEACAM1 presented by lymphocytes and CEACAM1 presented by cancer cells.

A “neutralizing antibody” as used herein refers to a molecule having an antigen-binding site to a specific receptor or ligand target capable of reducing or inhibiting (blocking) activity or signaling through a receptor, as determined by in vivo or in vitro assays, as per the specification.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. Monoclonal Abs may be obtained by methods known to those skilled in the art. For example, the monoclonal antibodies to be used in accordance with the present invention may be made by the hybridoma method first described by Kohler et al., Nature 1975, 256, 495, or may be made by recombinant DNA methods (see, e.g., U.S. Pat. No. 4,816,567). The “monoclonal antibodies” may also be isolated from phage antibody libraries using the techniques described in Clackson et al., Nature 1991, 352, 624-628 or Marks et al., J. Mol. Biol., 1991, 222:581-597, for example.

The term “plurality” as used herein refers to two or more of the object specified.

The mAbs may be of any immunoglobulin class including IgG, IgM, IgE, IgA. A hybridoma producing a mAb may be cultivated in vitro or in vivo. High titers of mAbs can be obtained in vivo production where cells from the individual hybridomas are injected intraperitoneally into pristine-primed BALB/c mice to produce ascites fluid containing high concentrations of the desired mAbs. Monoclonal Abs of isotype IgM or IgG may be purified from such ascites fluids, or from culture supernatants, using column chromatography methods well known to those of skill in the art.

The term “antigenic determinant” or “epitope” according to the invention refers to the region of an antigen molecule that specifically reacts with particular antibody. An “antigen” is a molecule or a portion of a molecule capable of eliciting antibody formation and being bound by an antibody. An antigen may have one or more than one epitope. An antigen according to the present invention is a CEACAM1 protein or a fragment thereof.

Antibodies, or immunoglobulins, comprise two heavy chains linked together by disulfide bonds and two light chains, each light chain being linked to a respective heavy chain by disulfide bonds in a “Y” shaped configuration. Proteolytic digestion of an antibody yields Fv (Fragment variable) and Fc (fragment crystalline) domains. The antigen binding domains, Fab, include regions where the polypeptide sequence varies. The term “F(ab′)₂” represents two Fab′ arms linked together by disulfide bonds. The central axis of the antibody is termed the Fc fragment. Each heavy chain has at one end a variable domain (VH) followed by a number of constant domains (CH). Each light chain has a variable domain (VL) at one end and a constant domain (CL) at its other end, the light chain variable domain being aligned with the variable domain of the heavy chain and the light chain constant domain being aligned with the first constant domain of the heavy chain (CH1). The variable domains of each pair of light and heavy chains form the antigen-binding site. The domains on the light and heavy chains have the same general structure and each domain comprises four framework regions, whose sequences are relatively conserved, joined by three hypervariable domains known as complementarity determining regions (CDR1-3). These domains contribute specificity and affinity of the antigen-binding site. The isotype of the heavy chain (gamma, alpha, delta, epsilon or mu) determines immunoglobulin class (IgG, IgA, IgD, IgE or IgM, respectively). The light chain is either of two isotypes (kappa, κ or lambda, λ) found in all antibody classes.

The term “a monoclonal antibody to human CEACAM1 as used herein refer to full immunoglobulin molecules, e.g. IgMs, IgDs, IgEs, IgAs or IgGs, antigen-binding-domains of such immunoglobulin molecules, e.g. Fab-fragments, Fab′-fragments, F(ab)2-fragements, chimeric F(ab)₂ or chimeric Fab′ fragments, chimeric Fab-fragments or isolated VH- or CDR-regions, and known isoforms and modifications of immunoglobulins, e.g. single-chain antibodies or single chain Fv fragments (scAB/scFv) or bispecific antibody constructs, capable of binding to their indicated targets. The term “anti-human-CEACAM1 antibody” as used herein refers to antibodies capable of binding to their indicated targets, wherein these targets are of human origin.

The terms “antigen-binding fragment of an antibody” and “antigen-binding fragment” as interchangeably used herein, refer to one or more fragments of an antibody that retains the ability to bind specifically to the disclosed antigen. For example, the antigen-binding fragment may include, but not limited to, Fab fragment, F(ab′)₂ fragment, scFv fragment, dAb fragment, CDR-containing fragment or isolated CDR. Therefore, an antigen-binding fragment of a monoclonal antibody to human CEACAM1 may be e.g. an Fab fragment of a monoclonal antibody to human CEACAM1, or any molecule which mimics the sequences and structure of such an Fab fragment, without being directly obtained from a monoclonal antibody to human CEACAM1, e.g. by chemical or enzymatic cleavage.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CHI domains and one or more cysteine residues at the C-terminus of the CH1 domain; (v) the Fv fragment having the VL and VH domains of a single arm of an antibody; (vi) the dAb fragment (Ward et al., Nature 1989, 341, 544-546) which consists of a VH domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 1988, 242, 423-426; and Huston et al., PNAS (USA) 1988, 85,5879-5883); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 1993, 90, 6444-6448); (xi) “linear antibodies” comprising a pair of tandem Fd segments (VH-CH1-VH-CH1) which, together with complementary light chain polypeptides, form a pair of antigen binding regions (Zapata et al. Protein Eng., 1995, 8, 1057-1062; and U.S. Pat. No. 5,641,870).

By the term “single chain variable fragment (scFv)” it is meant a fusion of the variable regions of the heavy and light chains of immunoglobulin, linked together with a short (usually serine, glycine) linker. Single chain antibodies can be single chain composite polypeptides having antigen binding capabilities and comprising amino acid sequences homologous or analogous to the variable regions of an immunoglobulin light and heavy chain (linked VH-VL or single chain Fv (scFv)). Both VH and VL may copy natural monoclonal antibody sequences or one or both of the chains may comprise a CDR-FR construct of the type described in U.S. Pat. No. 5,091,513, the entire contents of which are incorporated herein by reference. The separate polypeptides analogous to the variable regions of the light and heavy chains are held together by a polypeptide linker Methods of production of such single chain antibodies, particularly where the DNA encoding the polypeptide structures of the VH and VL chains are known, may be accomplished in accordance with the methods described, for example, in U.S. Pat. Nos. 4,946,778, 5,091,513 and 5,096,815, the entire contents of each of which are incorporated herein by reference.

Single chain antibodies can be single chain composite polypeptides having antigen binding capabilities and comprising amino acid sequences homologous or analogous to the variable regions of an immunoglobulin light and heavy chain i e linked V_{H}-V_{L} or single chain Fv (scFv).

The term “molecule having the antigen-binding portion of an antibody” as used herein is intended to include not only intact immunoglobulin molecules of any isotype and generated by any animal cell line or microorganism, but also the antigen-binding reactive fraction thereof, including, but not limited to, the Fab fragment, the Fab′ fragment, the F(ab′)₂ fragment, the variable portion of the heavy and/or light chains thereof, Fab mini-antibodies (see WO 93/15210, WO 96/13583, WO 96/37621, the entire contents of which are incorporated herein by reference), dimeric bispecific mini-antibodies (see Muller et al., 1998) and chimeric or single-chain antibodies incorporating such reactive fraction, as well as any other type of molecule or cell in which such antibody reactive fraction has been physically inserted, such as a chimeric T-cell receptor or a T-cell having such a receptor, or molecules developed to deliver therapeutic moieties by means of a portion of the molecule containing such a reactive fraction. Such molecules may be provided by any known technique, including, but not limited to, enzymatic cleavage, peptide synthesis or recombinant techniques.

The term “antigen” as used herein refers to a molecule or a portion of a molecule capable of eliciting antibody formation and/or being bound by an antibody. An antigen may have one or more than one epitope. For example, the protein CEACAM1 is considered an antigen by the present invention. In preferred embodiments, the antigens are human antigens.

The present invention further provides, in another aspect, a method of treating a patient having cancer, comprising the step of administering to the patient a pharmaceutical composition comprising an inhibitor of a kinase selected from the group consisting of B-Raf kinase mutant, a MEK1 kinase and a MEK2 kinase, and a pharmaceutical composition comprising a monoclonal antibody to human CEACAM1 or an antigen-binding fragment thereof, wherein the cancer cells express a B-Raf kinase mutant, thereby treating the cancer.

The term “treating cancer” as used herein, refers to administering therapeutic effective amounts of agents such as antibodies and/or B-Raf inhibitors and/or MEK1 inhibitors and/or MEK2 inhibitors to a patient diagnosed with cancer, to inhibit the further growth of malignant cells in the patient, to inhibit the spread of the malignant cells in the patient, and/or to cause the death of malignant cells in the patient. Thus, in certain embodiments, treating cancer means attenuating tumor progression, inhibiting the spread of the malignant cells in the patient, causing the death of malignant cells in the patient and any combination thereof. Each possibility represents a separate embodiment of the invention. In certain embodiments, treating cancer means attenuating tumor progression, causing the death of malignant cells in the patient, or both. In some embodiments, treatment comprises reducing the size, maintaining the size or slowing the growth of a primary or metastatic tumor. Each possibility represents a separate embodiment of the present invention.

The term “preventing cancer” refers to avoiding the appearance, lowering the incidence or minimizing the severity of cancer. It also refers to avoiding or delaying the progression of cancer from a lower stage (e.g. stage III, primary tumor) to a higher stage (e.g. stage IV, metastasis). In some embodiments, prevention comprises avoiding, postponing, or minimizing the appearance of a primary or a metastatic tumor. Each possibility represents a separate embodiment of the present invention.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The terms “cancer” and “cancerous” refer to or describe the physiological condition in mammals that is typically characterized by unregulated cell growth. The term “anti-neoplastic composition” refers to a composition useful in treating cancer comprising at least one active therapeutic agent capable of inhibiting or preventing tumor growth or function, and/or causing destruction of tumor cells. Therapeutic agents suitable in an anti-neoplastic composition for treating cancer include, but not limited to, chemotherapeutic agents, radioactive isotopes, toxins, cytokines such as interferons, and antagonistic agents targeting cytokines, cytokine receptors or antigens associated with tumor cells.

The phrase “cancer cells express a B-Raf kinase mutant” as used herein refers to cancer cells of the patient expressing or comprising at least one mutant B-Raf kinase protein.

The present invention further provides, in another aspect, a kit comprising an inhibitor of a kinase selected from the group consisting of a B-Raf kinase mutant, a MEK1 kinase and a MEK2 kinase, and a monoclonal antibody to human CEACAM1 or an antigen-binding fragment thereof.

The present invention further provides, in an aspect, the kit described above, for use in treating cancer. The present invention further provides, in an aspect, the kit described above, for use in preparing a medicament for treating cancer.

The term “kit” as used herein, refers to a combination of reagents and other materials. It is contemplated that the kit may include reagents such as antibodies, antibody mixtures, buffers, diluents and other aqueous solutions, and/or one or more storage vials or other containers. It is not intended that the term “kit” be limited to a particular combination of reagents and/or other materials.

Further, the present invention provides, in an aspect, a method of diagnosing resistance to an inhibitor of a kinase selected from the group consisting of a B-Raf kinase mutant, a MEK1 kinase and a MEK2 kinase in a cancer patient treated by the kinase inhibitor, comprising the steps of obtaining a biopsy sample from the patient, determining the level of CEACAM1 in cancer cells of the biopsy sample, and comparing the level of CEACAM1 in cancer cells of the biopsy sample to a predetermined threshold, wherein the cancer cells express a B-Raf kinase mutant, and wherein a level of CEACAM1 in cancer cells of the biopsy sample higher than the predetermined threshold is indicative of resistance to the kinase inhibitor in the patient.

The present invention further provides, in a related aspect, a method of diagnosing resistance to an inhibitor of a kinase selected from the group consisting of a B-Raf kinase mutant, a MEK1 kinase and a MEK2 kinase in a cancer patient treated by the kinase inhibitor, comprising the steps of determining the level of CEACAM1 in cancer cells of a biopsy sample obtained or derived from the patient, and comparing the level of CEACAM1 in cancer cells of the biopsy sample to a predetermined threshold, wherein the cancer cells express a B-Raf kinase mutant, and wherein a level of CEACAM1 in cancer cells of the biopsy sample higher than the predetermined threshold is indicative of resistance to the kinase inhibitor in the patient.

The terms “biopsy sample” and “sample” are interchangeable, and generally refer to a sample of a body fluid, to a sample of isolated cells or to a sample of cells from a tissue or an organ. Cells, tissue or organ samples may be obtained from any tissue or organ by, e.g., a biopsy. Isolated cells may be obtained from the tissues or organs by separating techniques such as trypsinization, centrifugation or cell sorting.

The diagnosis methods of the invention will in general be carried out in-vitro or ex-vivo. In particular, the references to the term “biological sample” are intended to indicate that the diagnosis methods are not in general carried out on the human or animal body, unless otherwise indicated. In particular, the term “obtained from” may comprise receiving a sample from an agent acting on behalf of the subject, e.g. receiving a sample from a doctor, nurse, hospital, medical center, etc. As used herein, the term “derived from” shall be taken to indicate that a specified sample is obtained from a particular source albeit not necessarily directly from that source.

Depending on the type of mutation in B-Raf, the B-Raf kinase activity towards MEK may vary, as most of the B-Raf mutants stimulate enhanced B-Raf kinase activity toward MEK. In certain embodiments, the B-Raf kinase inhibitor attenuates or prevents the phosphorylation of MEK1 or MEK2 by the B-Raf kinase mutant. In certain embodiments, the B-Raf kinase inhibitor attenuates or prevents the dimerization of the B-Raf kinase mutant.

The RAS/RAF/MEK/ERK pathway is critical in regulating cell proliferation, differentiation and apoptosis. The RAF proteins play an essential role in growth factor-mediated signal transduction from the cell surface to nucleus. B-Raf is the main isoform of RAF family that relays the signal transduction from RAS to MEK. Growth factors bind to and activate their receptor tyrosine kinases (RTKs) on cell membrane, which in turn convert RAS from an inactive GDP-bound to an active GTP-bound state. The binding of RAS-GTP and B-Raf recruits B-Raf to the cell membrane, where B-Raf is phosphorylated at Thr 599 and Ser 602 with enhanced serine/threonine kinase activity. The activated B-Raf leads to phosphorylation and activation of MEK1/2, which in turn activates ERK1/2 to phosphorylate downstream targets in the cytoplasm and nucleus (An et al., Scientific Reports, 2013, Vol. 3). Many known amino-acid mutations, such as V600E, allow B-Raf to signal independently of upstream cues. In certain embodiments, the B-Raf kinase mutant is constitutively active.

In certain embodiments, the B-Raf kinase mutant comprises a mutation in a position selected from the group consisting of V600, G469, D594, K601, G466, L597, G464, M117, I326, K439, T440, V459, R462, I463, F468, K475, N581, E586, D587, F595, G596, T599, R682, A728, and any combination thereof. Each possibility represents a separate embodiment of the present invention. In certain embodiments, the B-Raf kinase mutant comprises a mutation selected from the group consisting of V600E, V600D, V600G, V600K, V600M, and V600R. Each possibility represents a separate embodiment of the present invention. In certain embodiments, the B-Raf kinase mutant comprises the mutation V600E.

In certain embodiments, the B-Raf kinase inhibitor is selected from the group consisting of vemurafenib, dabrafenib, sorafenib, LGX818, RAF265, CEP-32496, XL281, RO5212054, and any combination thereof. In certain embodiments, the B-Raf kinase inhibitor is vemurafenib.

Activated B-Raf phosphorylates and activates MEK1 and/or MEK2, which in turn activate ERK1 (MAPK1) and/or ERK2 (MAPK2). In certain embodiments, the MEK1 kinase inhibitor attenuates or prevents the phosphorylation of MAPK by the MEK1 kinase. In certain embodiments, the MEK2 kinase inhibitor attenuates or prevents the phosphorylation of MAPK by the MEK2 kinase.

In certain embodiments, the MEK1 kinase inhibitor is selected from the group consisting of seulmetinib, trametinib, binimetinib, cobimetinib, PD-325901, PD-184352, TAK-733, PD-98059, SL-327, U-0126, BAY-869766, PD-198306, PD-184161, AS-703026, PD-318088, and any combination thereof. Each possibility represents a separate embodiment of the invention. In certain embodiments, the MEK1 kinase inhibitor is seulmetinib.

In certain embodiments, the MEK2 kinase inhibitor is selected from the group consisting of trametinib, binimetinib, cobimetinib, PD-325901, PD-184352, SL-327, U-0126, BAY-869766, PD-198306, PD-184161, AS-703026, PD-318088, and any combination thereof. In certain embodiments, the MEK2 kinase inhibitor is trametinib.

When the process of developing a specific antibody involves generation in a non-human immune system (such as that in mice), the protein sequences of the antibodies produced are partially distinct from homologous antibodies occurring naturally in humans, and are therefore potentially immunogenic when administered to human patients. Thus, to avoid immunogenicity when administered to a human patient, in some embodiments, the monoclonal antibody to human CEACAM1 is a human, humanized or chimeric monoclonal antibody. Each possibility represents a separate embodiment of the invention.

The term “human antibody”, as used herein, is intended to include antibodies having variable and constant regions derived from human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs. However, the term “human antibody”, as used herein, is not intended to include antibodies in which CDR sequences derived from the germline of another mammalian species, such as a mouse, have been grafted onto human framework sequences. In certain embodiments, the term “human antibody” refers to an isolated human antibody, i.e. isolated from a human donor. In certain embodiments, the term “human antibody” refers to a human antibody isolated from a hybridoma cell line. In certain embodiments, the term “human antibody” refers to a recombinant human antibody, i.e. produced by recombinant DNA technology.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 1996 14,309-314; Sheets et al. PNAS (USA), 1998, 95, 6157-6162); Hoogenboom and Winter, J. Mol. Biol., 1991, 227, 381; Marks et al., J. Mol. Biol., 1991, 222, 581). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al, Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

The term “recombinant human antibody”, as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor, L. D., et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “humanized antibody” as used herein refers is intended to include antibodies that have their CDRs (complementarity determining regions) derived from a non-human species immunoglobulin and the remainder of the antibody molecule derived mainly from a human immunoglobulin. The term “chimeric antibody” as used herein refers is intended to include antibodies that have their CDRs (complementarity determining regions) derived from a non-human species immunoglobulin and the remainder of the antibody molecule derived mainly from a human immunoglobulin.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance In general, the humanized antibody comprises substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also comprises at least a portion of an immunoglobulin constant region (Fc), typically that of a human immunoglobulin. For further details, see Jones et al., Nature 1986, 321, 522-525; Riechmann et al., Nature 1988, 332, 323-329; and Presta, Curr. Op. Struct. Biol., 1992 2, 593-596.

The term “recombinant human antibody”, as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor, L. D., et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the monoclonal antibody to human CEACAM1 is a human, humanized or chimeric monoclonal antibody. Each possibility represents a separate embodiment of the invention.

In certain embodiments, the monoclonal antibody to human CEACAM1 or the antigen-binding fragment thereof is capable of binding with an affinity of at least about 10⁻⁸M to human CEACAM1. In certain embodiments, the monoclonal antibody to human CEACAM1 or the antigen-binding fragment thereof is capable of binding with an affinity of at least about 5×10⁻⁷M to at least one of human CEACAM3 and human CEACAM5.

In some embodiments, the monoclonal antibody to human-CEACAM1, or a fragment, derivative or analog thereof comprises heavy chain CDR1 having the sequence set forth in SEQ ID NO: 7, heavy chain CDR2 having the sequence set forth in SEQ ID NO: 8 and heavy chain CDR3 having the sequence set forth in SEQ ID NO: 9. Each possibility represents a separate embodiment of the present invention.

In some embodiments, the monoclonal antibody to human-CEACAM1, or a fragment, derivative or analog thereof comprises heavy chain CDR1 having the sequence set forth in SEQ ID NO: 13, heavy chain CDR2 having the sequence set forth in SEQ ID NO: 14 and heavy chain CDR3 having the sequence set forth in SEQ ID NO: 15. Each possibility represents a separate embodiment of the present invention.

In some embodiments, the monoclonal antibody to human-CEACAM1, or a fragment, derivative or analog thereof comprises light chain CDR1 having the sequence set forth in SEQ ID NO: 10, light chain CDR2 having the sequence set forth in SEQ ID NO: 11 and light chain CDR3 having the sequence set forth in SEQ ID NO: 12. Each possibility represents a separate embodiment of the present invention.

In some embodiments, the monoclonal antibody to human-CEACAM1, or a fragment, derivative or analog thereof comprises light chain CDR1 having the sequence set forth in SEQ ID NO: 16, light chain CDR2 having the sequence set forth in SEQ ID NO: 17, and light chain CDR3 having the sequence set forth in SEQ ID NO: 18.

In some embodiments, the monoclonal antibody to human-CEACAM1, or a fragment, derivative or analog thereof has CDR sequences set forth in SEQ ID NOs: 13, 14, 15, 16, 17, and 18.

In some embodiments, the monoclonal antibody to human-CEACAM1, or a fragment, derivative or analog thereof has CDR sequences set forth in SEQ ID NOs: 7, 8, 9, 10, 11, and 12.

In some embodiments, the monoclonal antibody to human-CEACAM1, or a fragment, derivative or analog thereof, wherein the derivative or analog has at least 90% sequence identity with an antigen-binding portion of the monoclonal antibody.

In some embodiments, the monoclonal antibody to human-CEACAM1, or a fragment, derivative or analog thereof comprises a heavy chain variable domain sequence having a sequence set forth in SEQ ID NO: 26, or an analog or derivative thereof having at least 97% sequence identity with the heavy chain sequence.

In some embodiments, the monoclonal antibody to human-CEACAM1, or a fragment, derivative or analog thereof comprises a light chain variable domain sequence having a sequence set forth in SEQ ID NO: 28, or an analog or derivative thereof having at least 97% sequence identity with the light chain sequence. Each possibility represents a separate embodiment of the present invention.

In some embodiments, the monoclonal antibody to human-CEACAM1, or a fragment, derivative or analog thereof comprises a heavy chain variable domain having a sequence set forth in SEQ ID NO: 26 and a light chain variable domain having a sequence set forth in SEQ ID NO: 28, or an analog or derivative thereof having at least 97% sequence identity with the antibody or fragment sequence. Each possibility represents a separate embodiment of the present invention.

In some embodiments, the monoclonal antibody to human-CEACAM1, or a fragment, derivative or analog thereof comprises: (i) a framework sequence selected from the group consisting of: mouse IgG2a, mouse IgG2b, mouse IgG3, human IgG1, human IgG2, human IgG3; and (ii) six CDRs having sequences set forth in SEQ ID NOs: 13, 14, 15, 16, 17, and 18; or six CDRs having sequences set forth in SEQ ID NOs: 7, 8, 9, 10, 11, and 12; and analogs and derivatives thereof having at least 97% sequence identity with the CDR sequences, wherein the monoclonal antibody or fragment binds with an affinity of at least about 5×10⁻⁷M to at least two CEACAM subtypes. Each possibility represents a separate embodiment of the present invention.

In some embodiments, the monoclonal antibody to human-CEACAM1, or an antigen-binding fragment thereof is a chimeric monoclonal antibody. In some embodiments, the chimeric antibody to human-CEACAM1, or an antigen-binding fragment thereof comprises human derived constant regions selected from the group consisting of: human IgG1, human IgG2, and human IgG3. Each possibility represents a separate embodiment of the present invention.

In some embodiments, the chimeric monoclonal antibody to human-CEACAM1, or a fragment, derivative or analog thereof comprises the six CDRs having sequences set forth in SEQ ID NOs: 13, 14, 15, 16, 17, and 18; or the six CDRs having sequences set forth in SEQ ID NOs: 7, 8, 9, 10, 11, and 12; and analogs and derivatives thereof having at least 95% sequence identity with the CDR sequences, wherein the monoclonal antibody binds with an affinity of at least about 10⁻⁸M to CEACAM1. Each possibility represents a separate embodiment of the present invention.

In some embodiments, the chimeric monoclonal antibody to human-CEACAM1, or a fragment, derivative or analog thereof comprises a constant region subclass of human IgG1 subtype. Each possibility represents a separate embodiment of the present invention.

In some embodiments, the chimeric monoclonal antibody to human-CEACAM1, or a fragment, derivative or analog thereof comprises a heavy chain sequence set forth in SEQ ID NO: 30. Each possibility represents a separate embodiment of the present invention. In some embodiments, the chimeric monoclonal antibody to human-CEACAM1, or a fragment, derivative or analog thereof comprises a light chain sequence set forth in SEQ ID NO: 31. Each possibility represents a separate embodiment of the present invention. In some embodiments, the chimeric monoclonal antibody to human-CEACAM1, or a fragment, derivative or analog thereof comprises a heavy chain sequence set forth in SEQ ID NO: 30, and light chain sequence set forth in SEQ ID NO: 31. Each possibility represents a separate embodiment of the present invention.

In some embodiments, the monoclonal antibody to human-CEACAM1, or a fragment, derivative or analog thereof is a monoclonal antibody which recognizes CEACAM1 produced from DNA sequences of the heavy and light chains contained in a plasmid deposited on Sep. 28, 2011 under ATCC Accession Number PTA-12130. Each possibility represents a separate embodiment of the present invention.

In some embodiments, the monoclonal antibody to human-CEACAM1, or a fragment, derivative or analog thereof is a monoclonal antibody which recognizes CEACAM1, or a fragment thereof comprising at least the antigen-binding portion, which is capable of binding the same epitope on the CEACAM1 molecule to which a monoclonal antibody having the six CDR sequences set forth in SEQ ID NOs: 7, 8, 9, 10, 11 and 12, or the six CDR sequences set forth in SEQ ID NOs: 13, 14, 15, 16, 17 and 18, binds. Each possibility represents a separate embodiment of the present invention.

In some embodiments, the monoclonal antibody to human-CEACAM1, or a fragment, derivative or analog thereof is reactive with an epitope within residues 17-29 and 68-79 of human CEACAM1 having the sequences VLLLVHNLPQQLF (SEQ ID NO:32) and YPNASLLIQNVT (SEQ ID NO:33) respectively. Each possibility represents a separate embodiment of the present invention.

In some embodiments, the epitope on the CEACAM1 molecule to which the monoclonal antibody binds is an epitope comprising amino acid residues within the sequences VLLLVHNLPQQLF (SEQ ID NO: 32) and YPNASLLIQNVT (SEQ ID NO: 33). In some embodiments, the epitope on the CEACAM1 molecule to which the monoclonal antibody binds comprises at least four amino acids of the sequence VLLLVHNLPQQLF (SEQ ID NO: 32). In some embodiments, the epitope on the CEACAM1 molecule to which the monoclonal antibody binds is an epitope within sequences VLLLVHNLPQQLF (SEQ ID NO: 32) and PNASLLI (SEQ ID NO: 34).

In certain embodiments, the monoclonal antibody to human CEACAM1 is selected from the group consisting of CM-24, 26H7, 5F4, TEC-11, 12-140-4, 4/3/17, COL-4, F36-54, 34B1, YG-C28F2, D14HD11, b18.7.7, DU-ADM HEA81, B1.1, CLB-gran-10, F34-187, T84.1, B6.2, B1.13, YG-C94G7, 12-140-5, scFv DIATHIS1, TET-2, antigen-binding fragments thereof, and any combination thereof. Each possibility represents a separate embodiment of the invention. In certain embodiments, the monoclonal antibody to human CEACAM1 or the antigen-binding fragment thereof has a heavy-chain CDR1 comprising a sequence set forth in SEQ ID NO: 1, a heavy-chain CDR2 comprising a sequence set forth in SEQ ID NO: 2 a heavy-chain CDR3 comprising a sequence set forth in SEQ ID NO: 3, a light-chain CDR1 comprising a sequence set forth in SEQ ID NO: 4, a light-chain CDR2 comprising a sequence set forth in SEQ ID NO: 5 and a light-chain CDR3 comprising a sequence set forth in SEQ ID NO: 6.

Many types of cancer are associated with mutations in the BRAF gene, or with the aberrant function of B-Raf kinase mutants. For example, Davies and coworkers (Davies et al., Nature, 2002, Vol. 417, pages 949-954) identified BRAF mutations in 14 cancer cell lines and primary tumors. Garnett and Marais labeled BRAF is a human oncogene and reviewed its role in cancer (Garnett and Marais, Cancer Cell, 2004, Vol. 6, pages 313-319). In certain embodiments, the pharmaceutical compositions described above are for use in treating a cancer selected from the group consisting of melanoma, thyroid, colon, ovarian, liver, sarcoma, stomach, glioma, carcinoma, breast, ependymoma and lung cancer. Each possibility represents a separate embodiment of the invention. In certain embodiments, the pharmaceutical compositions described above are for use in treating melanoma. In certain embodiments, the pharmaceutical compositions described above are for use in treating stage III cancer. In certain embodiments, the pharmaceutical compositions described above are for use in treating stage IV cancer.

According to the surprising findings of the present invention, it is favorable to treat B-Raf-mutated cancers with a combination of inhibitors of the RAF/MEK pathway and antibodies to human CEACAM1. Without being bound to any theory or mechanism, it is hypothesized that the compositions of the present invention generate a “two-punch” combination, in which the administration of RAF/MEK inhibitors to cancer patients promotes cancer cell death accompanied by lowered levels of CEACAM1 expression within cancer cells of the patient, while the sensitivity of the cancer cells to these inhibitors cytotoxicity is maintained by interaction with anti-CEACAM1 antibodies, which prevent, delay or attenuate the appearance of RAF/MEK inhibitor-resistant cells. Further, the presence of antibodies to human CEACAM1 prevents, delays or attenuates the cancer cells from relaying an immuno-suppressive signal to cytotoxic T cells through the CEACAM1^{cancer cell}/CEACAM1^{T cell} pathway.

In certain embodiments, the kinase inhibitor and the monoclonal antibody to human CEACAM1 or the antigen-binding fragment thereof are administered simultaneously. In certain embodiments, the kinase inhibitor and the monoclonal antibody to human CEACAM1 or the antigen-binding fragment thereof are comprised in the same pharmaceutical composition. In certain embodiments, the kinase inhibitor and the monoclonal antibody to human CEACAM1 or the antigen-binding fragment thereof are comprised in at least two separate pharmaceutical compositions.

In certain embodiments, the kinase inhibitor and the monoclonal antibody to human CEACAM1 or the antigen-binding fragment thereof are administered separately. In certain embodiments, the kinase inhibitor is administered prior to the administration of the monoclonal antibody to human CEACAM1 or the antigen-binding fragment thereof. In certain embodiments, the monoclonal antibody to human CEACAM1 or the antigen-binding fragment thereof is administered prior to the administration of the kinase inhibitor.

In certain embodiment, the B-Raf kinase inhibitor attenuates or prevents the phosphorylation of MEK1 or MEK2 by the B-Raf kinase in the cancer cells of the cancer patient. In certain embodiments, the B-Raf kinase inhibitor attenuates or prevents the dimerization of the B-Raf kinase mutant in the cancer cells of the cancer patient.

The cancer stage grouping system uses numerals I, II, III, and IV to describe the progression of cancer. In stage I, cancers are localized to one part of the body. Stage I cancer can be surgically removed if small enough. In stage II, cancers are locally advanced. Stage II cancer can be treated by chemotherapy, radiation therapy, or surgery. In stage III, cancers are also locally advanced. Whether a cancer is designated as Stage II or Stage III can depend on the specific type of cancer; the specific criteria for Stages II and III therefore differ according to diagnosis. Stage III can be treated by chemotherapy, radiation therapy, or surgery. In stage IV, cancers have often metastasized, or spread to other organs or throughout the body. Stage IV cancer can be treated by chemotherapy, radiation therapy, or surgery. In certain embodiment, the method described above further comprises the step of a surgical resection of the primary tumor. In certain embodiment, the method described above further comprises the step of administering a chemotherapy agent or an additional immunomodulator to the patient. In certain embodiment, the method described above further comprises the step of exposing the patient to irradiation.

In the method of diagnosing resistance to an inhibitor of a kinase in a cancer patient provided by the present invention, the level of CEACAM1 in the cancer cells must be compared to a predetermined threshold, which stands in correlation with inhibitor-naïve subjects. It is the relative difference in CEACAM1 expression levels which is indicative of resistance. In certain embodiment, the predetermined threshold is the level of CEACAM1 in cells of a corresponding biopsy sample obtained from a corresponding tissue of a healthy control subject. In certain embodiment, the predetermined threshold is the level of CEACAM1 in cells of a prior biopsy sample obtained from the same tissue of the patient before the patient was treated with the kinase inhibitor. In some embodiments, the biopsy sample is obtained from a nevus of the patient. In some embodiments, the biopsy sample is obtained from a primary tumor of the patient. In some embodiments, the biopsy sample is obtained from a metastatic tumor of the patient.

The following examples are presented in order to more fully illustrate some embodiments of the invention. They should, in no way be construed, however, as limiting the broad scope of the invention.

### EXAMPLES

### Example 1

### CEACAM1 Expression Correlates with the Presence of B-Raf Mutations in Cancer Cells

Biopsy samples from 24 Melanoma cancer patients were tested for CEACAM1 expression levels and for BRAF genotype by flow cytometry and real time PCR, respectively.

Cell staining was performed by standard FACS staining with 0.1 μg of a murine monoclonal antibody to human CEACAM1 (described in WO/2010/125571) for 30 minutes on ice, followed by secondary FITC-conjugated goat anti mouse IgG. Total RNA was extracted from 1.0*10⁶ melanoma cells with TRIZOL. cDNA was generated with standard reverse transcriptase. Real time PCR was performed with CEACAM1-specific primers and compared to glyceraldehyde 3-phosphate dehydrogenase (GAPDH) tested as a control. Cycle threshold (Ct) values above 36 were considered as minimal or no expression. B-Raf genotype was determined by direct sequencing of amplified fragment from genomic DNA that contains the codon for position 600 of the B-Raf protein. Statistical analysis of the correlation between CEACAM1 expression (negative or positive) with the mutation status (positive=V600E, or negative=wild type) was performed by Fisher exact test.

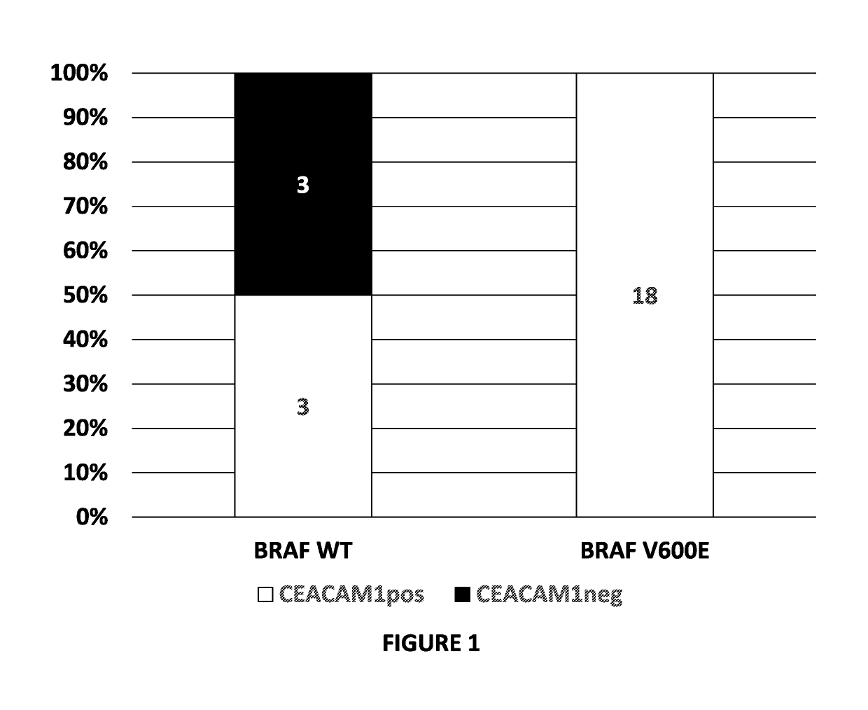

Table 1 summarizes the results of GAPDH expression, CEACAM 1 expression and B-Raf mutations obtained from the 24 patients featured in FIG. 1.

TABLE 1
Analysis of melanoma patients.

| | | | | | B-Raf | |
|---|---|---|---|---|---|---|
| | | GAPDH | CEACAM1 | CEACAM1 | muta- | Corre- |
| # | Sample | (Avg Ct) | (Avg Ct) | expression | tion | lation* |
| 1 | mel001 | 18.34 | 25.103 | + | + | + |
| 2 | mel003 | 17.01 | 27.6 | + | + | + |
| 3 | mel005 | 15.102 | 25.658 | + | + | + |
| 4 | mel008 | 19.124 | 21.502 | + | + | + |
| 5 | mel009 | 15.764 | 23.224 | + | − | − |
| 6 | mel002 | 15.193 | 25.287 | + | + | + |
| 7 | mel004 | 18.136 | 20.02 | + | − | − |
| 8 | mel013 | 17.668 | 24.279 | + | + | + |
| 9 | mel014 | 18.45 | 23.24 | + | + | + |
| 10 | mel015 | 20.238 | 24.74 | + | + | + |
| 11 | mel019 | 16.662 | 20.984 | + | + | + |
| 12 | mel020 | 17.264 | 30.115 | − | − | + |
| 13 | mel026 | 19.096 | 30.158 | − | − | + |
| 14 | mel039 | 18.06 | 25.17 | + | + | + |
| 15 | mel041 | 18.789 | 23.131 | + | + | + |
| 16 | mel042 | 16.5 | 25.54 | + | + | + |
| 17 | mel045 | 16.134 | 23.265 | + | + | + |
| 18 | mel046 | 15.398 | 20.749 | + | + | + |
| 19 | mel054 | 17.51 | 30.026 | − | − | + |
| 20 | mel067 | 16.829 | 22.972 | + | + | + |
| 21 | mel071 | 16.242 | 25.316 | + | + | + |
| 22 | mel072 | 18.386 | 26.505 | + | + | + |
| 23 | mel073 | 17.062 | 26.195 | + | + | + |
| 24 | mel076 | 22.247 | 26.55 | + | − | − |

*Correlation between CEACAM1 expression and B-Raf mutation.

The data presented in Table 1 and FIG. 1 demonstrates the statistically significant correlation between B-Raf V600E and expression of CEACAM1 (P value<0.01, Fisher's exact test). More specifically, whereas only 50% (3/6) of the melanoma cells having a wild type B-Raf, i.e a valine in position 600, expressed detectable levels of CEACAM1 (Ct of 36 and less), 100% (18/18) of the melanoma cells having a mutated B-Raf, i.e a glutamic acid in position 600, expressed detectable levels of CEACAM1 (Ct of 36 and less).

### Example 2

### B-Raf Inhibitors Selectively Inhibit the MAPK Pathway in B-Raf V600E Cancer Cells

MAPK activity was measured in B-Raf V600E (624mel, sk-mel-5) and B-Raf W.T. (076mel, 013mel) cell samples by immunoblotting for phosphorylated ERK1/2 (pERK, Thr202/Tyr204), total ERK1/2 and actin after 48 hours of exposure to vemurafenib (1 μM).

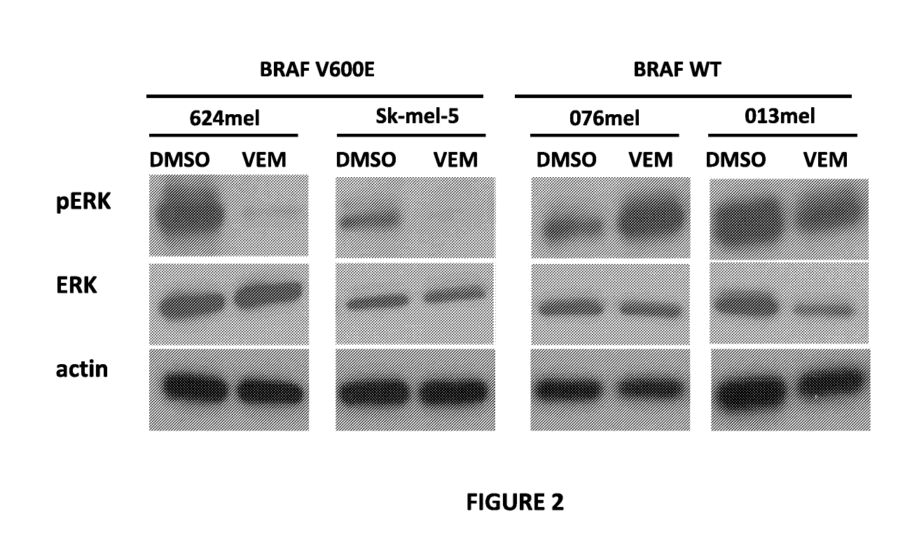

The data presented in FIG. 2 demonstrates that in B-Raf V600E cells vemurafenib almost completely abolishes the phosphorylation of ERK1/2, while in B-Raf W.T. cells vemurafenib has no substantial effect.

### Example 3

### CEACAM1 Extracellular Staining of Cancer Cells Treated with B-Raf or MEK Inhibitors

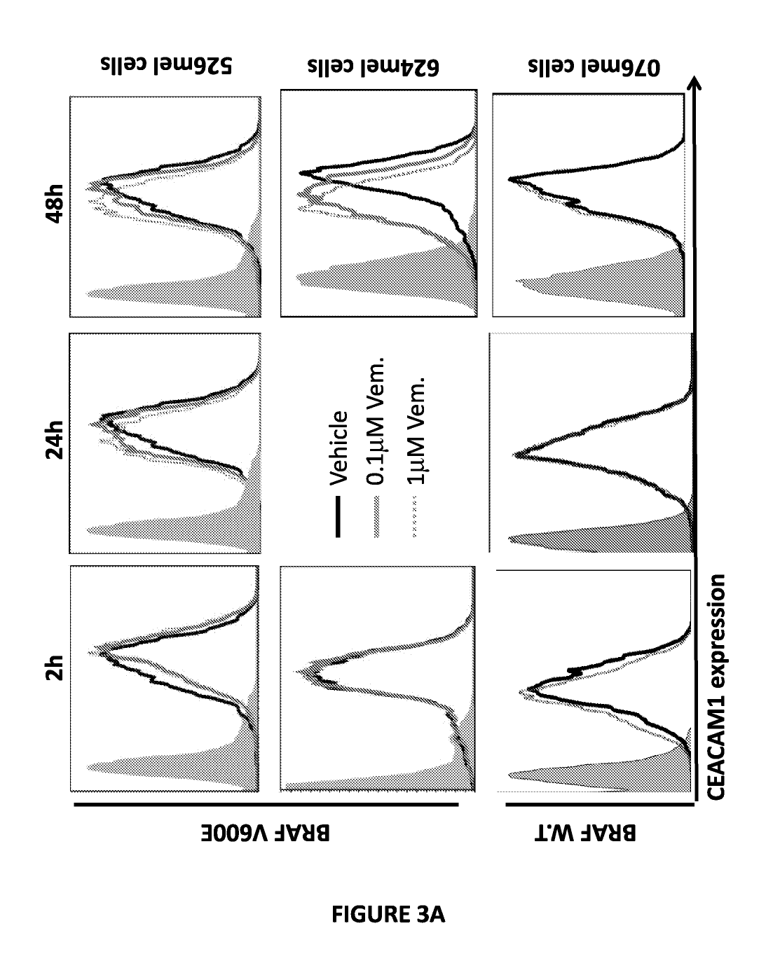

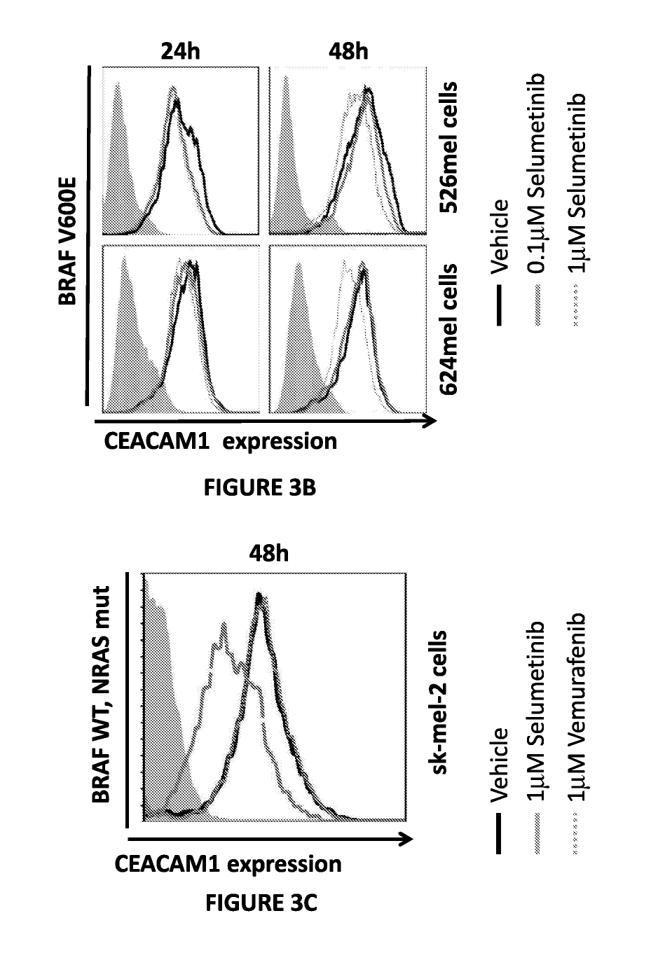

1.0*10⁶ cells of a B-Raf W.T. cell sample (076mel) and two B-Raf V600E cell samples (526mel, 624mel) were incubated with different concentrations of vemurafenib or selumetinib (0.1 μM or 1 μM) for 2 to 48 hours. At each time point, CEACAM1 expression on the cells was determined by FACS. Volume equivalents of DMSO (vehicle) were used as control. The data presented in FIG. 3A demonstrates that background staining (full gray histogram) was much lower than the control (0.1% DMSO, black line). The data further demonstrates that while 0.1 μM and 1 μM vemurafenib did not have any effect on CEACAM1 expression levels on cells having W.T. B-Raf (076mel), 0.1 μM (gray line) and 1 μM vemurafenib (gray dotted line) had a dose-dependent effect on CEACAM1 expression levels on cells having mutated B-Raf (526mel, 624mel). The data presented in FIG. 3B demonstrates that selumetinib had a similar effect to vemurafenib on cells having mutated B-Raf (526mel, 624mel).

The data presented in FIG. 3C demonstrates that while 1 μM selumetinib (gray dashed line) significantly decreased CEACAM1 expression levels on cells having W.T. B-Raf but mutated N-Ras (sk-mel-2), 1 μM vemurafenib (gray dotted line) had no effect on CEACAM1 expression levels. These results further support the regulation of CEACAM1 via the constitutively activated MAPK pathway, which is driven in this case by mutated N-Ras, and not by mutated B-Raf.

### Example 4

### Inhibitor-Resistant Cancer Cells Show Increase in CEACAM1 Expression and Restored Activity of MAPK Pathway

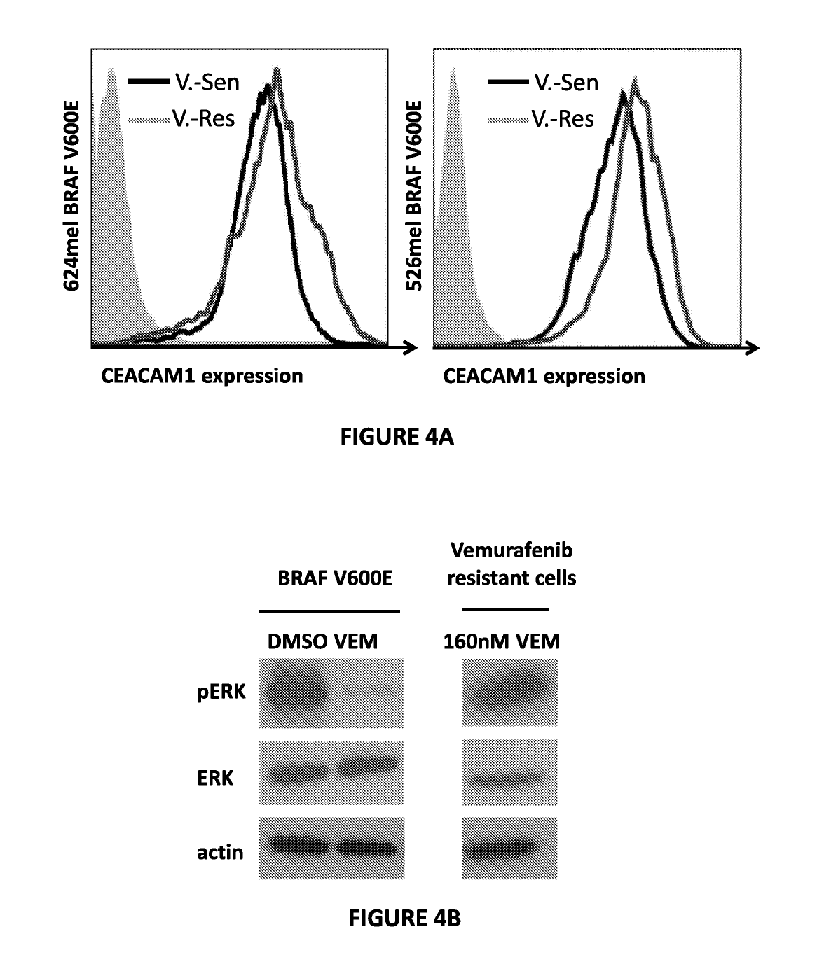

Two vemurafenib-sensitive B-Raf V600E cell samples (526mel, 624mel) and vemurafenib-resistant cell lines derived therefrom were incubated for 2 days with 1 μM vemurafenib. Cells were then analyzed for CEACAM1 protein expression by FACS as described above. Vemurafenib-resistant cell lines were generated by gradual increase of the inhibitor's concentration in culture, up to 0.32 μM. The data presented in FIG. 4A demonstrates that vemurafenib-resistant cell lines (gray line) expressed higher levels of CEACAM1 than vemurafenib-sensitive cell lines (black line).

MAPK activity was measured in vemurafenib-sensitive and vemurafenib-resistant B-Raf V600E (624mel) cell samples by immunoblotting for phosphorylated ERK1/2 (pERK, Thr202/Tyr204), total ERK1/2 and actin after 24 hours of exposure to 160 nM vemurafenib. The data presented in FIG. 4B demonstrates that in vemurafenib-sensitive B-Raf V600E cells vemurafenib almost completely abolishes the phosphorylation of ERK1/2, wherein MAPK activity was practically uninterrupted by vemurafenib in vemurafenib-resistant B-Raf V600E cells.

### Example 5

### Inhibitor-Resistant Cancer Cells Upregulate CEACAM1 Expression

B-Raf V600E 526mel melanoma cells were cultured in the presence of 1 μM vemurafenib. Cultivation was performed in RPMI 1640 supplemented with 1 mM Na-Pyruvate, 1 mM Pen-Strep, 1 mM L-Glutamine, 1 mM non-essential amino acids, and 10% heat inactivated fetal calf serum. Initial vemurafenib concentration was 0.01 of the determined IC₅₀ (0.64 nM). Each week, the concentration was doubled up to 5 times the IC₅₀ (320 nM), to generate vemurafenib-resistant melanoma cells. Cells were then tested for CEACAM1 expression using MRG1 (murine antibody to human CEACAM1) in flow cytometry as described above. Total RNA was extracted with TRIZOL and cDNA was generated with a reverse transcriptase, according to routine protocols.

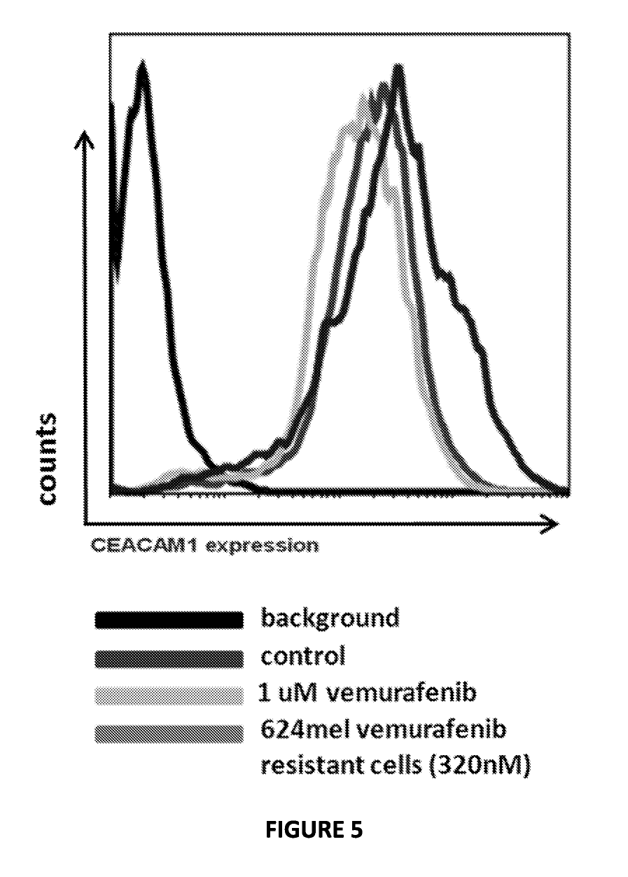

The data presented in FIG. 5 demonstrates that while vemurafenib down regulates CEACAM1 expression in B-Raf V600E melanoma cells, these cells upregulate CEACAM1 expression levels upon acquiring resistance to vemurafenib. It is important to note that CEACAM1 levels in B-Raf V600E melanoma cells after acquiring resistance to vemurafenib are higher than CEACAM1 levels in untreated (vemurafenib-naive) B-Raf V600E melanoma cells.

### Example 6

### Inhibitor-Resistant Cancer Cells Upregulate Expression of Both Types of CEACAM1

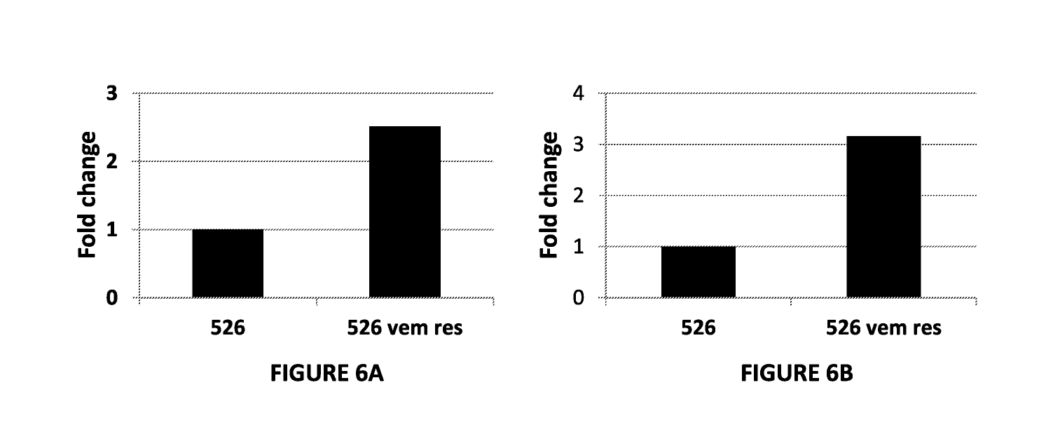

The vemurafenib-sensitive and vemurafenib-resistant B-Raf V600E 526mel melanoma cells of Example 5 were tested for the type of CEACAM1 over-expressed upon acquiring resistance to vemurafenib by qPCR. The data presented in FIG. 6 demonstrates that the expression of both types of CEACAM1, CEACAM1-long (A) and CEACAM1-short (B) is about three-fold upregulated in vemurafenib-resistant cells compared to vemurafenib-sensitive cells.

### Example 7

### B-Raf/MEK Inhibitors Increase T-Cell Induced Cytotoxicity

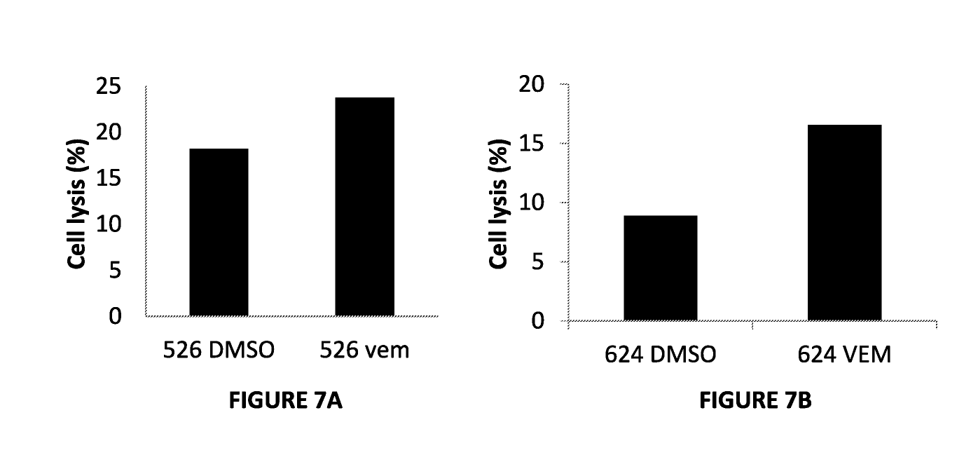

Two vemurafenib-sensitive B-Raf V600E cell samples (526mel, 624mel) were tested for viability in the presence of cytotoxic T cells, with or without 1 μM vemurafenib. Melanoma cells were pre-incubated with 1 μM vemurafenib and then co-incubated overnight with HLA-A2 matched antigen-matched T cells in effector-target ratio of 5:1. Cell killing was determined by LDH release. The data presented in FIG. 7 demonstrates that vemurafenib significantly sensitizes melanoma cells to cytotoxic T cells.

### Example 8

### B-Raf/MEK Inhibitors and Antibodies to CEACAM1 Increase T-cell Induced Cytotoxicity to Cancer Cells In-Vitro

Two vemurafenib-sensitive B-Raf V600E cell samples (526mel, 624mel) are tested for viability before and after being treated by the agents specified in Table 2 below.

TABLE 2
Therapeutic combinations for treating cancer cells in-vitro.

| | Cytotoxic | B-Raf | MEK | Antibody to |
|---|---|---|---|---|
| # | T cells | inhibitor | inhibitor | human CEACAM1 |
| 0 | − | − | − | − |
| 1 | + | − | − | − |
| 2 | + | + | − | − |
| 3 | + | + | − | + |
| 4 | + | − | + | − |
| 5 | + | − | + | + |
| 6 | + | + | + | + |

### Example 9

### B-Raf/MEK Inhibitors and Antibodies to CEACAM1 Increase the Immune Response Against Cancer Cells In-Vivo

Human cancer cells are transplanted subcutaneously in nude mice, mice are randomized into 7 treatment groups (n=20) as specified in Table 3 below. The existence and volume of primary tumors and metastasis is determined before and after treatment.

TABLE 3
Therapeutic combinations for treating cancer in-vivo.

| | Cytotoxic | B-Raf | MEK | Antibody to |
|---|---|---|---|---|
| # | T cells | inhibitor | inhibitor | human CEACAM1 |
| 0 | − | − | − | − |
| 1 | + | − | − | − |
| 2 | + | + | − | − |
| 3 | + | + | − | + |
| 4 | + | − | + | − |
| 5 | + | − | + | + |
| 6 | + | + | + | + |

### Example 10

### B-Raf/MEK Inhibitors and Antibodies to CEACAM1 Increase the Immune Response Against Cancer Cells In-Vivo

Cancer patients (n=10) are treated by the agents specified in Table 4 below. The existence and volume of primary tumors and metastasis is determined before and after treatment.

TABLE 4
Therapeutic combinations for treating cancer in-vivo.

| | Cytotoxic | B-Raf | MEK | Antibody to |
|---|---|---|---|---|
| # | T cells | inhibitor | inhibitor | human CEACAM1 |
| 0 | − | − | − | − |
| 1 | + | − | − | − |
| 2 | + | + | − | − |
| 3 | + | + | − | + |
| 4 | + | − | + | − |
| 5 | + | − | + | + |
| 6 | + | + | + | + |

### Example 11

### Inhibitor-Resistant Cancer Cells Become Immune Resistant in a CEACAM1-Dependent Manner

Naïve melanoma cells, vemurafenib- or selumetinib-resistant V600E melanoma cells, and melanoma cells isolated after 48 hour incubation with 1 μM of vemurafenib or selumetinib, are tested in tumor infiltrating lymphocytes (TILs) cytotoxicity assays. Each cell type is pre-incubated in the presence or absence of various concentrations of an anti-CEACAM1 antibody (e.g. MRG1) up to 5 μg/ml. Then, the viability of the cells is determined by standard techniques, and cells are co-incubated for 5 hours with melanoma-specific human TILs in an effector to target ratio which varies from 1:1 to 10:1. Then, the viability of the cells is determined again.

### Example 12

### B-Raf-W.T. Cancer Cells Transfected with B-Raf V600E Enhance CEACAM1 Expression and Become Immune Resistant in a CEACAM1-Dependent Manner

Melanoma cells bearing wild type B-Raf are stably transfected with a plasmid encoding for V600E B-Raf to simulate the effect of the emergence of mutated B-Raf. Transfection of an empty vector serves as negative control. Overexpression is verified with Western blot for B-Raf. The presence of mutated B-Raf is verified with sequencing of PCR amplified cDNA. Cells are tested for CEACAM1 expression by FACS as described above. Cytotoxicity assays as described above are performed, to evaluate the cytotoxicity of melanoma-specific T cells on B-Raf V600E-transfected cells. The influence of a CEACAM1 blocking monoclonal antibody (e.g. MRG1) on the cytotoxicity is determined.

1-54. (canceled)

55. A pharmaceutical composition comprising:
(i) an inhibitor of a kinase selected from the group consisting of a B-Raf kinase mutant, a MEK1 kinase and a MEK2 kinase; and
(ii) a monoclonal antibody to human CEACAM1 or an antigen-binding fragment thereof.

56. The pharmaceutical composition of claim 55, wherein the B-Raf kinase mutant comprises a mutation in a position selected from the group consisting of V600, G469, D594, K601, G466, L597, G464, M117, 1326, K439, T440, V459, R462, I463 F468, K475, N581, E586, D587, F595, G596, T599, R682, A728, and any combination thereof.

57. The pharmaceutical composition of claim 56, wherein the B-Raf kinase mutant comprises a mutation selected from the group consisting of V600E, V600D, V600G, V600K, V600M, and V600R.

58. The pharmaceutical composition of claim 55, wherein the kinase inhibitor is a B-Raf kinase inhibitor selected from the group consisting of vemurafenib, dabrafenib, sorafenib, LGX818, RAF265, CEP-32496, XL281, RO5212054, and any combination thereof.

59. The pharmaceutical composition of claim 55, wherein the kinase inhibitor is MEK1 or MEK2 kinase inhibitor selected from the group consisting of seulmetinib, trametinib, binimetinib, cobimetinib, PD-325901, PD-184352, TAK-733, PD-98059, SL-327, U-0126, BAY-869766, PD-198306, PD-184161, AS-703026, PD-318088, and any combination thereof.

60. The pharmaceutical composition of claim 55, wherein the monoclonal antibody to human CEACAM1 is a human, humanized or chimeric monoclonal antibody capable of binding with an affinity of at least about 10⁻⁸M to human CEACAM1.

61. The pharmaceutical compositions of claim 60, wherein the monoclonal antibody to human CEACAM1 or the antigen-binding fragment thereof is capable of binding with an affinity of at least about 5×10⁻⁷M to at least one of human CEACAM3 and human CEACAM5.

62. The pharmaceutical compositions of claim 60, wherein the monoclonal antibody to human CEACAM1 or the antigen-binding fragment thereof has a heavy-chain CDR1 comprising a sequence set forth in SEQ ID NO: 1, a heavy-chain CDR2 comprising a sequence set forth in SEQ ID NO: 2 a heavy-chain CDR3 comprising a sequence set forth in SEQ ID NO: 3, a light-chain CDR1 comprising a sequence set forth in SEQ ID NO: 4, a light-chain CDR2 comprising a sequence set forth in SEQ ID NO: 5 and a light-chain CDR3 comprising a sequence set forth in SEQ ID NO: 6.

63. A method of treating a patient having cancer, comprising the step of administering to the patient:
(i) a pharmaceutical composition comprising an inhibitor of a kinase selected from the group consisting of B-Raf kinase mutant, a MEK1 kinase and a MEK2 kinase; and
(ii) a pharmaceutical composition comprising a monoclonal antibody to human CEACAM1 or an antigen-binding fragment thereof;
wherein the cancer cells express a B-Raf kinase mutant; thereby treating the cancer.

64. The method of claim 63, wherein the kinase inhibitor, and the monoclonal antibody to human CEACAM1 or the antigen-binding fragment thereof, are administered simultaneously.

65. The method of claim 64, wherein the kinase inhibitor, and the monoclonal antibody to human CEACAM1 or the antigen-binding fragment thereof, are comprised in the same pharmaceutical composition.

66. The method of claim 63, wherein the kinase inhibitor, and the monoclonal antibody to human CEACAM1 or the antigen-binding fragment thereof, are administered separately.

67. The method of claim 63, wherein the kinase inhibitor is a B-Raf kinase inhibitor selected from the group consisting of vemurafenib, dabrafenib, sorafenib, LGX818, RAF265, CEP-32496, XL281, RO5212054, and any combination thereof.

68. The method of claim 63, wherein the kinase inhibitor is a MEK1 or MEK2 kinase inhibitor selected from the group consisting of seulmetinib, trametinib, binimetinib, cobimetinib, PD-325901, PD-184352, TAK-733, PD-98059, SL-327, U-0126, BAY-869766, PD-198306, PD-184161, AS-703026, PD-318088, and any combination thereof.

69. The method of claim 63, wherein the monoclonal antibody to human CEACAM1 is a human, humanized or chimeric monoclonal antibody capable of binding with an affinity of at least about 10⁻⁸M to human CEACAM1.

70. The method of claim 69, wherein the monoclonal antibody to human CEACAM1 or the antigen-binding fragment thereof is capable of binding with an affinity of at least about 5×10⁻⁷M to at least one of human CEACAM3 and human CEACAM5.

71. The method of claim 60, wherein the monoclonal antibody to human CEACAM1 or an antigen-binding fragment thereof has a heavy-chain CDR1 comprising a sequence set forth in SEQ ID NO: 1, a heavy-chain CDR2 comprising a sequence set forth in SEQ ID NO: 2 a heavy-chain CDR3 comprising a sequence set forth in SEQ ID NO: 3, a light-chain CDR1 comprising a sequence set forth in SEQ ID NO: 4, a light-chain CDR2 comprising a sequence set forth in SEQ ID NO: 5 and a light-chain CDR3 comprising a sequence set forth in SEQ ID NO: 6.

72. The method of claim 63, wherein the cancer is selected from the group consisting of melanoma, thyroid, colon, ovarian, liver, sarcoma, stomach, glioma, carcinoma, breast, ependymoma and lung cancer.

73. The method of claim 72, wherein the cancer is a stage III cancer or a stage IV cancer.

74. A method of diagnosing resistance to an inhibitor of a kinase selected from the group consisting of a B-Raf kinase mutant, a MEK1 kinase and a MEK2 kinase in a cancer patient treated by the kinase inhibitor, comprising the steps of:
(i) obtaining a biopsy sample from the patient;
(ii) determining the level of CEACAM1 in cancer cells of the biopsy sample; and
(iii) comparing the level of CEACAM1 in cancer cells of the biopsy sample to a predetermined threshold selected from the level of CEACAM1 in cells of a corresponding biopsy sample obtained from a corresponding tissue of a healthy control, and the level of CEACAM1 in cells of a prior biopsy sample obtained from the same tissue of the patient before the patient was treated with the kinase inhibitor,
wherein the cancer cells express a B-Raf kinase mutant, and wherein a level of CEACAM1 in cancer cells of the biopsy sample higher than the predetermined threshold is indicative of resistance to the kinase inhibitor in the patient.
